(19) United States
(12) Patent Application Publication
(10) Pub. No.: US 2010/0168037 A1
(43) Pub. Date: Jul. 1, 2010

(54) PEPTIDES COMPRISING AROMATIC D-AMINO ACIDS AND METHODS OF USE

(75) Inventor: Byron E. ANDERSON, Morton Grove, IL (US)

Correspondence Address: BARNES & THORNBURG LLP, P.O. BOX 2786, CHICAGO, IL, 60690-2786, US

(73) Assignee: BIO SCIENCE INTERNATIONAL, INC., Germantown, WI (US)

(21) Appl. No.: 12/697,087

(22) Filed: Jan. 29, 2010

Related U.S. Application Data
(63) Continuation-in-part of application No. 10/612,298, filed on Jul. 2, 2003, now Pat. No. 7,655,602.
(60) Provisional application No. 60/394,176, filed on Jul. 3, 2002.

(51) Int. Cl. A61K 38/06; C40B 30/04; C40B 40/10; A61K 38/07; A61K 38/08; C07K 7/06

(52) U.S. Cl. 514/16; 506/9; 506/18; 514/17; 514/18; 530/329; 530/330; 530/331

(57) ABSTRACT

Disclosed are D-peptides and libraries of D-peptides comprising aromatic D-amino acids. Also disclosed are methods for identifying small D-peptides comprising aromatic D-amino acids that bind to proteins of interest

### CROSS-REFERENCE TO RELATED APPLICATIONS

This application is a continuation-in-part of co-pending U.S. Ser. No. 10/612,298, filed Jul. 3, 2003, which claims priority from U.S. Provisional Patent Application Ser. No. 60/394,176, filed Jul. 3, 2002. These applications are hereby incorporated by referenced in their entireties.

### BACKGROUND

One approach to identifying a molecule with potential therapeutic value is to assess the ability of that molecule to bind to a protein having an important biological activity, because the activity of the protein may be altered by its binding to a molecule that does not normally serve as a substrate or ligand for the protein.

The biological activities of many proteins are modulated by binding of the proteins to other molecules. For example, lectins are a class of proteins whose activities are affected by binding to carbohydrate ligands, including monosaccharides and oligosaccharides. Lectins are found in most living organisms, including mammals and other animals, prokaryotes, plants and even viruses. Lectins are involved in many important functions, including, for example, active transport and chemotaxis in bacteria, establishing viral infections, mediating leukocyte-endothelial cell recognition, mediating attachment of bacteria or viruses to other cells, and recognizing normal or pathologic glycoproteins and polysaccharides. Because lectins are involved in important biological activities, they are attractive targets for drug therapy.

### SUMMARY OF THE DISCLOSURE

Combinatorial libraries and methods to prepare then and use them to identify molecules (peptides) with potential therapeutic value by the molecules ability to bind to a protein having an important biological function (activity) are disclosed. The D-configuration amino acids are preferred because peptides identified, are to be used for therapeutic purposes. The D-configuration peptides are expected to have longer half-lives in living organisms compared to L-configuration peptides. The hydrophobic nature of peptides containing 3 or more aromatic residues also predicts a partition of the peptides in a living organism to proteins in the blood, such as albumin, also resulting in longer half-lives in the blood circulation. Combinatorial libraries including high percents of the members of the libraries containing 3 or more of the aromatic D-amino acids, D-phenylalanine, D-tyrosine and D-tryptophan, methods of identifying particular aromatic enriched peptides that bind to proteins of therapeutic and/or diagnostic significance, and the methods of determining affinities of the peptides to particular proteins are described. Unexpected findings are that the aromatic peptides are highly specific to particular proteins, and that the particular peptides identified do not show non-specific binding to other proteins or to a wide range of proteins. There are a large number of uses for the D-aromatic enriched peptides with binding activities to proteins of diagnostic and therapeutic importance.

In one aspect, D-peptides includes sequences of from three to seven D-amino acid residues (i.e., tri- to hepta-peptides), wherein three of the amino acid residues are selected from the group including D-tryptophan, D-tyrosine and D-phenylalanine.

In another aspect, a D-peptide includes a pentapeptide sequence selected from the following group Xaa₁YYFF, Xaa₁FYFF, Xaa₁YFFF, Xaa₁FFYF, Xaa₁YFFY, Xaa₁YFYF, Xaa₁FFFF, Xaa₁FYYF, FXaa₁FFF, YFXaa₁FF, Xaa₁FWXaa₂Y, Xaa₁FXaa₂WY, Xaa₁Xaa₂FFW, Xaa₁FFFY, FFFFXaa₁, YXaa₁YFF, YXaa₁FFY, Xaa₁FF Xaa₂Xaa₃, Xaa₁WYFF, Xaa₁FXaa₂FF, Xaa₁YXaa₂FF, Xaa₁FFYXaa₂, Xaa₁FFXaa₂F, Xaa₁Xaa₂Xaa₃YY, Xaa₁Xaa₂Xaa₃FF, Xaa₁FYWF, Xaa₁Xaa₂FYY, Xaa₁YYFY, Xaa₁FYXaa₂Y, WXaa₁FFF, Xaa₁FFFXaa₂, Xaa₁YYYY, FXaa₁WFF, WXaa₁FWXaa₂, WFXaa₁FXaa₂, FWXaa₁FF, FXaa₁FFY, Xaa₁Xaa₂WXaa₃Y, FFWXaa₁Y, FXaa₁WXaa₂Xaa₃, YYXaa₁YY, FFFXaa₁F, YFYFXaa₁, YWXaa₁FF, WXaa₁YXaa₂F, WXaa₁YFXaa₂, WXaa₁FFXaa₂, FFFXaa₁W, FWFXaa₁Xaa₂, FYXaa₁YF, FWXaa₁Xaa₂Xaa₃, FXaa₁YYW, FXaa₁YYXaa₂, FWXaa₁WY, FFWYW, FXaa₁Xaa₂FXaa₃, FYWXaa₁Y, FYWXaa₁W, FXaa₁YFXaa₂, FWWYF, FYYYXaa₁, and FFXaa₁WW, wherein Xaa₁, Xaa₂, and Xaa₃ are amino acids of the D- or L-configuration independently selected from the group D, E, K, R, H, N, Q, S, T, G, A, V, L, I, M, and P.

In another aspect, a combinatorial library is described including a plurality of D-peptides, wherein each D-peptide includes a sequence of from three to seven D-amino acid residues. The sequences of at least 68% of the D-peptides comprise at least three amino acid residues independently selected from the group including D-tryptophan, D-tyrosine, and D-phenylalanine.

Small peptides are synthesized, preferably composed of D-configuration aromatic amino acids, and with spacer amino acids, and preferably of 3 to 7 amino acid residues, which exhibit biologic activities of therapeutic significance. Combinatorial libraries preferably are tri-, tetra-, penta-, hexa-, and hepta-peptides in size. A pentapeptide combinatorial library was synthesized and tested. Pentapeptides are intermediate in size of the tri- to hepta-peptide libraries, and the size of a pentapeptide is equivalent in size to the average space of natural ligands for protein binding sites.

Penta-, hexa- and hepta-peptides are sizes that effect sufficient non-covalent bonds with a protein binding site to yield high affinities. The numbers of different peptide sequences in such combinatorial libraries with 3 or more aromatic residues are 2,133 for the pentapeptide library, 12,825 for the hexapeptide combinatorial library, and 66,393 for the heptapeptide combinatorial library, where the libraries utilize 5 amino acids including D-phenylalanine, D-tyrosine, D-tryptophan, D-alanine and glycine. The latter two amino acids are considered “spacers” in the peptide sequences and are expected to contribute little to the binding interactions with protein binding sites. The calculations of the 2,133, 12,825 and 66,393 numbers of peptides from the penta-, hexa- and hepta-peptide libraries, respectively, containing 3 or more aromatic D-amino acid residues are described herein.

In yet another aspect, a method for making a D-peptide that binds to a pre-selected protein, includes contacting a combinatorial library of D-peptides with the protein, detecting binding of the protein to a D-peptide to yield a bound D-peptide, identifying the bound D-peptide, and synthesizing the D-peptide.

In an aspect, a method for reducing toxicity of a toxin in a mammal exposed to the toxin includes delivering to the mammal a D-peptide of D-amino acids identified as binding to the toxin in an amount effective to reduce toxicity.

### DETAILED DESCRIPTION

Aromatic residues arranged closely in space, and preferably in a linear residue order, can mimic oligosaccharides in a functional sense, and be of a nature, structure and sequence to bind into carbohydrate binding sites of lectin type proteins. The aromatic residues may be of several chemical types and linked in a number of fashions to yield 3-dimensional structures that sterically can insert into carbohydrate binding sites, and are of a chemical nature to form a number of non-covalent bonding interactions definable by kinetic parameters. The aromatic constructs may be small peptides of D- or L-configurations, or mixtures of both D- and L-configuration amino acids, and composed of natural amino acids or permutations thereof. The data in the Examples section herein support that the small aromatic peptides are binding to aromatic residues which are characteristically found in carbohydrate binding sites. The aromatic residues may also form non-covalent binding interactions with other R groups in carbohydrate binding sites. Many proteins which bind to ligands of chemical types different then carbohydrate structures also utilize aromatic amino acid groups in such binding interactions. Examples of such non-carbohydrate binding proteins, the protective antigen of the anthrax toxin, TNFα and TGFβ are described in the Examples section.

The data in the Examples herein show that short peptide sequences of D-configuration amino acids containing preferably 3 to 5 aromatic residues (D-phenylalanine, D-tyrosine, D-tryptophan) bind to many proteins, including toxins, lectins, antibodies and cytokines, with high affinities and selectivity. For example, the binding affinities determined for some botulinum toxins and for the ricin toxin are in the low nM range, making such peptides feasible for therapeutic and diagnostic uses.

Two sequential screening steps are used to greatly increase the different peptide sequences used, and also to increase both affinities of binding as well as specificities of binding. In the first screening of a particular protein binding to members of the combinatorial peptide libraries, a small number of peptides are identified composed of the 3 or more aromatic residues and D-alanine and/or glycine, and the sequences of the identified peptides determined. The positions of D-alanine or glycine residues are thus apparent, and a secondary library is then made by permutating the D-alanine and/or glycine positions (keeping the aromatic residues at their identified positions) with all the other D-configuration amino acids, i.e., excluding phenylalanine, tyrosine, tryptophan, but including alanine and/or glycine as in the original sequence).

For a pentapeptide containing 3 aromatic residues and 2 residues of D-alanine and/or glycine as identified in the first screening, the secondary library consists of 16×16=256 members. In this way a very large number of potential different sequences are surveyed: for the pentapeptide library this 2 step method potentially covers a total of 75,843 pentapeptide sequences including 3 or more aromatic residues plus all other amino acids at the non-aromatic positions.

For the hexapeptide combinatorial library and the 2 step screening procedure, a total of 2,546,937 different hexapeptide sequences are surveyed of peptides containing 3 or more aromatic residues in the initial screening of the hexapeptide combinatorial library. For the heptapeptide combinatorial library, a total of 73,376,091 sequences can be surveyed. These numbers are large enough for expectations that the 2 step screening procedure will yield peptide sequences with high specificities because the R groups of the other D-amino acids may form a better 3-dimensional complementarity to the protein binding regions with probable additional non-covalent bond formations.

For the tri- and tetra-peptide libraries, the number of different peptides of 3 or 4 aromatic residues is low and therefore high specificity of most of such peptides binding to particular proteins is not as likely. However, such libraries and particular peptides, or mixtures thereof, are useful in affinity chromatography of complex mixtures of proteins, and possibly lipids.

Because of the hydrophobic nature of the aromatic amino acids, some amount of non-specific binding was expected with many different proteins. Surprisingly, the data in the Examples show this is not the case.

In an aspect, the ability of proteins of interest to bind to D-peptides including D-aromatic acids was evaluated with the expectation that D-peptides having therapeutic or diagnostic utility would be identified. As used herein, a D-peptide is a peptide including amino acids of D-configuration. In addition to D-amino acids, the D-peptides may further include one or more L-configuration amino acids. By proteins of interest, it is meant any protein having or suspected of having biological activity that may be altered by binding of a molecule D-peptide to the protein. As discussed herein, lectins mediate many important biological functions and therefore, are potentially useful targets in drug design. Other proteins of interest include, without limitation, protein toxins, such as those produced by various bacterial pathogens, and antibodies.

In order to test the ability of D-peptides to bind to pre-selected proteins of interest, combinatorial libraries of pentapeptides enriched in aromatic D-amino acid residues were synthesized and then tested for the ability to bind lectins, various protein toxins, various antibodies and other proteins. Those of skill in the art will appreciate that using methods disclosed herein, combinatorial libraries of short D-peptides ranging from three to seven amino acid residues in length are useful to identify a D-peptide that binds to other proteins of diagnostic and/or therapeutic interest.

For libraries of D-peptides having from three to seven amino acid residues, a library enriched in D-peptides including aromatic D-amino acids is one in which 68% or more of sequences in the library include three or more aromatic D-amino acid residues.

As described in the Examples, a pentapeptide library enriched in aromatic D-amino acids was constructed in a split synthesis method using four D-amino acids (alanine, phenylalanine, tyrosine, and tryptophan, or, using the one-letter codes for the amino acids, A, F, Y, and W, respectively) and glycine (G). Glycine is achiral and therefore, does not have D- or L-configurations. For a tripeptide aromatic combinatorial library, only the three aromatic D-amino acids are used as described below; for a tetrapeptide combinatorial library only the three aromatic D-amino acid residues plus G are used. As used herein, the A, F, Y and W amino acids, or other amino acids, are of the D-configuration, unless otherwise specified. For the sake of clarity in designating the amino acids with the one letter codes, the capital letter is used to designate the D-configuration amino acids, even though convention is to use the lower case letters. (The reason for using the capital letters to designate the D-configuration amino acids is that the lower case letters are often difficult to read and thus particular sequences may be misinterpreted; using the capital letters reduces the probably of confusion as to amino acid sequences.)

One wishing to create a library enriched in D-peptides including aromatic D-amino acids may do so using any suitable method. In the method described herein, 1.02% of the pentapeptides in the pentapeptide combinatorial library will contain no aromatic amino acids in the library made by the split synthesis method; 7.68% of the pentapeptides will contain one aromatic D-amino acid; 23.04% of the pentapeptides in the library will contain two aromatic D-amino acid residues; 34.56% of the pentapeptides will contain three aromatic D-amino acid residues; 25.92% of the pentapeptides will contained four aromatic D-amino acid residues; and 7.78% will contain five aromatic D-amino acid residues. Thus 68.26% of all peptides in the pentapeptide combinatorial library are sequences including three or more aromatic D-amino acid residues. For the synthesis of the hexapeptide and heptapeptide combinatorial libraries by the split synthesis method as described herein, the amino acids used are F, Y, W, A and G; for the synthesis of the tetrapeptide combinatorial library four of the amino acids are used, F, Y, W and G because the peptides consist of four amino acids and only one spacer, G, would therefore be used in order to obtain a combinatorial library with most of the sequences containing three or four aromatic D-amino acids; for the synthesis of the tripeptide combinatorial library, three amino acids are used, and only the three aromatic D-amino acids F, Y and W because each peptide has only three amino acid residues, and thus all peptides in the tripeptide combinatorial library would contain three aromatic D-amino acid residues.

The percents of D-peptides containing 3 or more aromatic residues in each combinatorial library can be readily calculated: For the tripeptide combinatorial library there are three aromatic residues possible at each of the three positions of the tripeptides. Because only the three aromatic residues are used in the construct of the library, there are a total of 27 (3 to the 3^{rd} power) different tripeptide sequences and all consist of 3 aromatic amino acid residues. For the tetrapeptide combinatorial library there are the three aromatic residues of F, Y and W, plus G used in the construct of the library. Thus there are a total of 256 (4 to the 4^{th} power) different tetrapeptide sequences and 189 of the total (73.8%) contain 3 or 4 aromatic amino acid residues. For the pentapeptide combinatorial library, as described above, there are a total of 3,125 (5 to the 5th power) different pentapeptide sequences of which 2133 (68.26%) contain 3, 4 or 5 aromatic amino acid residues. For the hexapeptide combinatorial library there are total of 15,675 (5 to the 6^{th} power) different hexapeptide sequences of which 12,825 (82.08%) contain 3, 4, 5 or 6 aromatic amino acid residues. For the heptapeptide combinatorial library there are a total of 78,125 (5 to the 7^{th} power) different heptapeptide sequences of which 66,393 (84.98%) contain 3, 4, 5, 6 or 7 aromatic amino acid residues.

For D-peptide sequences in which an amino acid residue may be selected from any one of a number of amino acids, the residue may be designated “Xaa₁”. In D-peptides having more than one amino acid residue selected from any one of a number of amino acids, such amino acid residues will be designated “Xaa₁”, “Xaa₂”, “Xaa₃”, etc.

Suitably, the D-peptides in a library may be attached to a solid support. In the Examples, a combinatorial library of pentapeptides enriched in aromatic D-amino acids was synthesized on TentaGel beads, each of which has a polystyrene core and, attached to the core, a plurality of polyoxyethylene arms, each arm having a primary amine at its free end. D-peptides were synthesized by sequential conjugation of each amino acid residue added to the D-peptide, using conventional standard D-peptide synthesis chemistry and a split synthesis protocol as described herein. The D-peptides thus constructed at the ends of the polyoxyethylene arms have free amino termini. The split synthesis method yields beads each of which includes multiple copies of a single D-peptide sequence.

Because the polyoxyethylene arms of the TentaGel beads are water soluble, the conformations of the attached D-peptides are determined primarily by thermodynamics and by their primary sequence. Also, being at the ends of the water soluble polyoxyethylene arms, the peptides are readily available for binding of proteins dissolved in water based buffers and incubated with the combinatorial libraries of peptides attached to the TentaGel beads. As one skilled in the art will appreciate, the D-peptide may be attached to any suitable support. For example, D-peptides including at least one lysine residue at the carboxyl terminus were synthesized and covalently coupled to maleic anhydride-coated 96-well polystyrene plates for use in binding assays.

Based on the results obtained in combinatorial library screenings, summarized in the Examples, the contribution of the aromatic amino acids F, Y, and W in the D-peptides is important for binding to proteins. Suitably, a D-peptide includes a sequence of from three to seven D-amino acid residues in length, which sequence includes at least three or more aromatic D-amino acid residues.

Although G and A were used as non-aromatic amino acids in the construction of the exemplified combinatorial D-peptide libraries described herein, libraries are not restricted to D-peptides or D-peptide libraries including G and A residues. As an example, it is specifically contemplated that additional D-peptides or D-peptide libraries are suitably generated by replacing G and/or A with any one of the remaining D-amino acids (i.e., D, E, K, R, H, N, Q, C, S, T, V, L, I, M, and P). For example, by replacing G and A with D-serine (S) and D-leucine (L), an additional library of 3125 members each may be constructed. Also as described herein, secondary combinatorial libraries are contemplated where the G and/or A positions of an identified peptide sequence are permutated with 16 other D-amino acids (keeping the positions of the aromatic D-amino acids). It is also contemplated that the G or A residues may be replaced with amino acids of the L-configuration producing libraries of mixed D- and L-configuration peptides.

Secondary combinatorial libraries may be made after identifying any particular D-peptide from the first screening of the combinatorial libraries of 4, 5, 6, or 7 residues. For example, a pentapeptide is identified as binding a particular protein of diagnostic or therapeutic interest which has 3 aromatic D-amino acids in its sequence plus 2 amino acids, D-Ala and/or Gly. A secondary combinatorial library may then be made wherein the latter two amino acid positions are permutated using all of the other D-configuration amino acids. This secondary library is then screened for binding of the same protein. It is expected that certain sequences in the secondary binding screen will exhibit higher affinities and specificities than the first identified peptide sequence containing D-Ala and/or Gly because the R groups of the other D-amino acids may form better 3-dimensional structural complementarily to the protein binding regions with probable additional non-covalent bond formations.

It is reasonably expected that a fourth aromatic D-configuration amino acid, D-histidine, may be incorporated into additional combinatorial libraries and screened for binding of selected proteins to members of such libraries. It is also reasonably expected that the aromatic residues (D-configuration of F, Y and/or W residues) of the combinatorial libraries or identified D-peptides from protein binding experiments may be replaced with “unusual” or “non-natural” amino acids of D- or L-configurations, e.g., D- or L-α-amino butyric acid, p-chloro-D-phenylalanine, p-chloro-L-phenylalanine, D-(2-naphthyl)alanine, or L-(2-naphthyl)alanine. Such unusual amino acids are commercially available as derivatives suitable for peptide syntheses. The library described in the Examples has D-peptides with the amino-terminus as a free amino group. Free amino groups may be derivatized, e.g., acetylated, and the resultant library of peptides tested for binding abilities to any protein of interest. A suitable library is constructed in the same manner except by eliminating the free amino group at the amino termini of the D-peptides. This may be accomplished by adding at the last step of the construction of the library the compounds acetic acid, propionic acid, 3-phenyl-propionic acid, 3-(4-hydroxy-phenyl)-propionic acid or 3-indole-propionic acid.

A D-peptide sequence identified as binding to a protein of interest may be used to design additional libraries by replacing the non-aromatic residues with other non-aromatic residues. For example, if a D-peptide having an A residue at a particular position is identified as binding to a protein, other combinatorial sublibraries may be readily constructed with permutations at the A position. A sublibrary including additional D-peptides may be constructed by replacing A with one of the amino acids not used in the construction of the original library. For a D-peptide sequence having G or A at two or more positions, one could replace the residues at 2 positions where a G or A residue is found with the other D-amino acids to create a new combinatorial sublibrary with 196 members (16×16=196, the 16 amino acids used are D-configurations of the natural amino acids except D-A, G, D-W, D-Y and D-F. Sublibraries thus created may be screened to identify members with different binding specificity or affinity for the protein of interest than the originally identified D-peptide. Certain D-peptide sequences in such combinatorial sublibraries may exhibit greater binding affinities and specificities because the R groups of the other D-amino acids may form a better 3-dimensional fit with the site of binding on the protein with consequent additional non-covalent bonds being formed.

An aromatic compound combinatorial library may also be constructed using building blocks that are not amino acids. For example, α-hydroxy- or β-hydroxy-carboxylic acids with aromatic constituents on the α- or β-carbons may be used and the individual carboxylic acids coupled to each other via formation of ester bonds. The combinatorial library could be built using the appropriate carboxylic analogues of G, A, F, Y and W (e.g., glycolic acid, lactic acid, phenyl-lactic acid, 3-(4-hydroxyphenyl)-lactic acid or 3-indole-lactic acid) using carbodiimide catalyzed couplings, and screened for binding to a protein of interest as described in the Examples.

A suitable combinatorial library may be built using β-amino acids composed of the appropriate analogues of the amino acids G, A, F, Y and W on TentaGel beads in the same manner as done for the D-configuration α-amino acids. Synthesis of D-peptides using β-amino acids analogues was described in Applella et al., (Nature, 387: 381-384, 1997), which is incorporated by reference herein.

A pre-selected protein used in screening the D-peptide combinatorial libraries may be any protein of interest, including lectins, protein toxins, or antibodies, for example. In the Examples below, the jack bean lectin (ConA), the garden pea lectin (PSA), and the lectin designated GSI-B4, as well as two anti-carbohydrate antibodies, were used to screen the D-peptide library for the ability to bind proteins. Competitive binding assays described in the Examples suggest that D-peptides may bind to carbohydrate binding sites. However, it should be understood that methods described are not limited only to those D-peptides that bind to carbohydrate binding sites.

In other Examples, proteins toxins, including botulinum toxins, ricin toxins, cholera toxin, and a component of the anthrax toxin, were screened for the ability to bind to D-peptides of the combinatorial library. It is of particular interest to identify molecules that can interact with toxins such as these because of the potential for biological warfare using toxins. For each toxin tested, D-peptides having the ability to bind to the proteins were identified.

Clostridium botulinum produces seven types of botulinum neurotoxins designated BoNT/A to BoNT/G. The toxins inhibit release of acetylcholine from the pre-synaptic neurons into the neuronal synapse, which may ultimately cause paralysis. Binding of the toxin to cells is required for toxicity. Blocking the binding of the botulinum toxins to the target cells, or blocking the protease activities of the neurotoxins, would prevent or reduce the pathogenic effects of the toxins.

In the Examples, D-peptides that bind to BoNT/A, BoNT/B or BoNT/E were identified. A mixture of three D-peptides having the ability to bind BoNT/A were administered to mice injected with the BoNT/A toxin. Preliminary data using live mice suggest that the D-peptides reduce toxicity of the BoNT/A toxin in mammals.

The botulinum toxin binding domain resembles other toxins, including the tetanus neurotoxin (TeNT) (Shapiro et al., J. Biol. Chem., 272, 30380-30386, 1997), diptheria toxin (Choe et al., Nature, 357, 216-222, 1992) and Pseudomonas aeriginosa exotoxin A (Allerud et al., Proc. Natl. Acad. Sci., 83, 1320-1324, 1986). It is therefore expected that D-peptides having the ability to bind to TeNT, diptheria toxin, and exotoxin A may also be identified using such libraries, and that such D-peptides may reduce toxicity of the toxins in a mammal.

Ricin is a plant cytotoxin composed of a cell surface binding domain (B) and an enzymatically active A domain with N-glycosidase activity (Lord et al., Semin. Cell Biol., 2, 15-22, 1991). The B domain binds to galactose residues of a cell surface and the A domain cleaves a single adenine from a conserved sequence of rRNA thus inactivating the ribosome and resulting in cell death. (Endo and Tsurugi, J. Biol. Chem., 263, 8735-8739, 1988). The identification of a D-peptide having the ability to bind to ricin may reduce binding of the toxin to cells or reduce its activity, thereby reducing toxicity.

The cholera toxin has one A subunit and five B subunits, and is similar in overall structure to the E. coli enterotoxin, the Shigella dysenteriae toxin and the Bordetella pertussis toxin. The cholera toxin binds to cell surface ganglioside GM₁ on the luminal surface of intestinal epithelial cells, where the A subunit is internalized and modifies guanine nucleotide-binding proteins involved in regulation of adenylate cyclase. Blocking the binding of the B subunit to the target cells will block A subunit internalization and reduce toxicity associated with the toxin.

The anthrax toxin has three components: the protective antigen (PA), lethal factor (LF) and edema factor (EF). The PA binds to the host cell surface receptor, is cleaved by a furin-like protease and the carboxy-terminal fragment heptamerizes and binds LF or EF (Milne et al., J. Biol. Chem., 269, 20607-20612, 1994; Elliott et al., Biochemistry, 39, 6706-6713, 2000). The EF and LF are translocated to the cytosol of the host cell, where EF activates an adenylate cyclase activity and LF, a protease, cleaves members of the mitogen-activated protein kinase family. Binding of a D-peptide to a component of the anthrax toxin may reduce toxicity.

In other Examples, antibodies were screened for the ability to bind to D-peptides in the D-peptide combinatorial library. One antibody tested was an antibody which binds to a carbohydrate epitope composed of the H and Ley carbohydrate sequences, which binds to an antigen of endothelial cells and inhibits activities associated with an angiogenic response (Szekanecz and Koch, Current Opinion.sub.—in Rheumatology, 13:202-208, 2001). The D-peptide identified as binding to the antibody may be used to study angiogenesis or to act as an agonist or antagonist of angiogenesis. A human antibody to an α-Gal epitope involved in the primate rejection response to transplanted porcine organs (Galili, Biochimie 83:557-563, 2001) was screened to identify D-peptides that bind to the antibody. Those D-peptides may be useful in blocking rejection mechanisms mediated by the human anti-α-Gal antibodies.

In other Examples, TNFα and TGFβ1 were screened for their ability to bind D-peptides in the D-peptide combinatorial library, and several D-peptide sequences were identified. TNFα and TGFβ1 are proteins involved in many cell signaling pathways (LaCuca and Gaspari, Dermatologic Clinics 19:617-635, 2001; Taylor, Current Opinion in Rheumatology 13:164-169, 2001; Massague, Nature Review Molecular Cell Biology 1:169-178, 2000; Letterio, Cytokine & Growth Factor Reviews 11:81-87, 2000). The D-peptides identified may be used to study signaling pathways or as possible therapeutic agents in pathologies in which TNFα and TGFβ1 are involved as mediators.

After a D-peptide has been identified as binding to a pre-selected protein according to the method, one of ordinary skill in the art can readily synthesize the D-peptide in sufficient quantity for further evaluation or for use as a therapeutic, which can be used to alter the activity of the pre-selected protein or, in the case of a protein toxin, reduce the toxicity of the toxin. The identified D-peptides may also be used as affinity reagents to purify any particular protein or to remove any particular protein from some biologic source material.

For those D-peptides intended for administration to a mammal, (e.g., a mammal exposed to a toxin), the D-peptides are suitably constructed or modified so as to enhance solubility. In the Examples below, D-peptides administered to mice were designed and synthesized to include three D-lysine residues at the C-terminal ends of the D-peptides to enhance solubility. From one to four D-lysine residues at the C-terminus would enhance solubility. Any amino acid residue tending to promote solubility may be included at the C-terminus, including R, D and/or E amino acids. The D-peptides may be derivatized at the C-terminus with substituents other than amino acids to promote solubility and/or to increase the half-life of the peptide conjugates in a biologic fluid such as blood of peptide conjugates administered to an animal. Such substituents may include a polyoxyethlene linker or polymer, or a compound containing multiple hydroxyl groups, such as a monosaccharide, oligosaccharide or polysaccharide. One or more of the D-peptides may be chemically coupled to a water soluble compound such as a polysaccharide or protein to promote solubility in water-based solvents or physiologic fluids. The D-peptides may be physically incorporated into or chemically coupled to structures such as liposomes in order to promote solubility in water-based physiologic fluids. More than one D-peptide may be coupled to a carrier molecule so as to multimerize the resulting conjugated compound for administration to a mammal with the potential effect of achieving a functional affinity (avidity) of the D-peptide multimer. More than one D-peptide identified as binding to a protein of interest may be coupled to a carrier compound to potentially achieve functional affinity effects. Additionally, one or more of the D-peptides may be conjugated to another peptide, protein or carbohydrate sequence (for example, the sialyl-lactose carbohydrate sequence known to have a binding site on the botulinum neurotoxin) in order to enhance binding of such conjugates to a protein of interest. Any of such peptide conjugates may also increase the half-life of the peptide administered to an animal with resultant increased therapeutic effects.

The polypeptide sequences described herein can be administered in any acceptable manner including orally, parenterally, nasally, by implant, and the like. Oral administration includes administration in tablets, suspension, implants, solutions, emulsions, capsules, powders, syrups, water composition, and the like. Nasal administration includes administering the compositions in sprays, solutions, and the like.

The therapeutic agents can be administered parenterally by injection or by gradual perfusion over time. Administration may be intravenously, intra-peritoneally, intramuscularly, subcutaneously, intra-cavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, intravenous vehicles including fluid and nutrient replenishers, electrolyte replenishers (such as those based on Ringer's dextrose), and the like. Preservatives and other additives may also be present, such as antimicrobials, anti-oxidants, chelating agents or inert gases and the like.

The actual dosage of a polypeptide sequences described, formulation, or composition will depend on many factors, including the size and health of an individual. However, the appropriate dosage may be determined by one of ordinary skill in the art. The following teachings, which are incorporated by reference, provide guidance: Spilker B., Guide to Clinical Studies and Developing Protocols, Raven Press Books, Ltd., New York, 1984, pp. 7-13, 54-60; Spilker B., Guide to Clinical Trials, Raven Press, Ltd., New York, 1991, pp. 93-101; Craig C., and R. Stitzel, eds., Modern Pharmacology, d. ed., Little, Brown and Co., Boston, 1986, pp. 127-33; T. Sleight, ed., Avery's Drug Treatment: Principles and Practice of Clinical Pharmacology and Therapeutics, 3d ed., Williams and Wilkins, Baltimore, 1987, pp. 50-56; R. Tallarida, R. Raffa and P. McGonigle, Principles in General Pharmacology, Springer-Verlag, New York, 1988, pp. 18-20. A polypeptide sequence of the invention may be conveniently administered in unit dosage form, and may be prepared by any of the methods well known in the pharmaceutical art, for example, as described in Remington's Pharmaceutical Sciences (Mack Pub. Co., Easton, Pa., 1990).

Physiologically acceptable and pharmaceutically acceptable excipients and carriers are well known to those of skill in the art. By “physiologically or pharmaceutically acceptable carrier” as used herein it is meant any substantially non-toxic carrier for administration in which a polypeptide sequences described will remain stable and bioavailable when used. For example, the polypeptide sequences can be dissolved in a liquid, or dispersed or emulsified in a medium in a conventional manner to form a liquid preparation or mixed with a semi-solid or solid carrier to form a paste, ointment, cream, lotion or the like.

Suitable carriers include water, petroleum jelly (VASELINE.RTM.), petrolatum, mineral oil, vegetable oil, animal oil, organic and inorganic waxes, such as microcrystalline, paraffin or ozocente wax, natural polymers, such as xanthanes, gelatin, cellulose, or gum arabic, synthetic polymers, alcohols, polyols, water and the like. A water miscible carrier composition that is substantially miscible in water can be used. Such water miscible carrier compositions can include those made with one or more ingredients set forth above but can also include sustained or delayed release carrier, including water containing, water dispersible or water soluble compositions, such as liposomes, microsponges, microspheres or microcapsules, aqueous base ointments, water-in-oil or oil-in-water emulsions, or gels.

The carrier can comprise a sustained release or delayed release carrier. The carrier may be any material capable of sustained or delayed release of the polypeptide sequence. The carrier is capable of releasing the polypeptide sequence when exposed to the environment of the area of intended delivery by diffusing or by release dependent on the degree of loading of the sequence to the carrier in order to obtain release of the polypeptide of the invention. Nonlimiting examples of such carriers include liposomes, microsponges, microspheres, matrices, or microcapsules of natural and synthetic polymers and the like. Examples of suitable carriers for sustained or delayed release in a moist environment include gelatin, gum arabic, xanthane polymers; by degree of loading include lignin polymers and the like; by oily, fatty or waxy environment include thermoplastic or flexible thermoset resin or elastomer including thermoplastic resins such as polyvinyl halides, polyvinyl esters, polyvinylidene halides and halogenated polyolefins, elastomers such as brasiliensis, polydienes, and halogenated natural and synthetic rubbers, and flexible thermoset resins such as polyurethanes, epoxy resins and the like. The sustained or delayed release carrier can be a liposome, microsponge, microsphere or gel. A pH balanced buffered aqueous solution for injection can be used. As one of skill in the art will appreciate, the preferred carrier will vary with the mode of administration. The compositions for administration usually contain from about 0.0001% to about 90% by weight of the polypeptide sequence compared to the total weight of the composition.

The D-peptide libraries may be useful in identifying D-peptides that may be used in affinity chromatographic purification of the pre-selected protein of interest. The D-peptides can readily be covalently coupled, using well-known chemistries, to any one of a number of suitable matrices used in chromatographic separations. The D-peptide matrices may be used to bind to the pre-selected protein from mixtures followed by elution and recovery of the protein.

The following nonlimiting examples are intended to be purely illustrative.

### Examples

### Peptide Library Design and Synthesis

A D-peptide combinatorial library was synthesized by Peptides International, Louisville, Ky. using a TentaGel S resin, NH₂ (“TentaGel beads”) following directions described herein. With the exception of glycine, which is an achiral molecule, all of the amino acid residues in the D-peptides are of the D configuration. The TentaGel beads have a polystyrene core with polyoxyethylene arms attached to the core; each arm has a primary amine functional group at its terminus. The resin contains 8.87.times.10.sup.5 beads/gram, an average bead diameter of 130 microns, 0.2-0.3 meq/gram capacity and 280-330 pmole of primary amine groups/bead capacity. The amino acids were conjugated to the resin and deprotected using standard D-peptide synthetic chemistries.

Amino acids are designated herein with the one-letter code. All amino acids are of the D-configuration unless otherwise noted. Glycine was attached to the resin to achieve about a 30% substitution of the available primary amine groups at the ends of the polyoxyethylene chains of the Tent-Gel beads. The amine groups to which glycine was not added were blocked by acetylation using acetic anhydride. A 30% substitution yields an average spacing of about 100 to 200 angstroms between D-peptides on the bead surface. The spacing was chosen to optimize binding of a single protein to a single D-peptide sequence, and to reduce the likelihood that steric hindrance will prevent a protein molecule from binding to a D-peptide or that a protein molecule will bind to more than one D-peptide.

Following blocking of the unreacted primary amine groups, the D-peptide combinatorial library was built by the split synthesis method (Lebl et al., Biopolymers (Peptide Science), 37, 177-198 (1995). The resin mixture was divided equally into five portions and one of G, A, F, Y or W was added by covalent coupling to one of the five portions of the G-substituted resin. The beads were then combined, again equally divided into five portions, and each portion was used in reactions in which one of G, A, F, Y or W was added in the separate reaction mixtures. The procedure was repeated for the five cycles to yield a combinatorial library of pentapeptide sequences attached to the G residues of the resin. Each bead contained multiple copies of a single D-peptide sequence. Because five amino acids were used at each of five amino acid adding steps, the resulting bead combinatorial library contains a total of 3125 different pentapeptide sequences, of which 2,133 of the sequences contain 3, 4 or 5 aromatic D-amino acids. Following the final amino acid addition, the resin batches were kept separate, which resulted in five sublibraries of 625 different sequences, designated G, A, F, Y, or W, according to the last amino acid added. Libraries of different length peptides are prepare in similar fashion.

Screening for Protein Binding to D-Peptides and Results of Binding Assays to the D-peptide Substituted Beads (Peptide-Beads)

In general, except as otherwise noted, proteins were screened for binding to the D-peptide beads as follows.

An aliquot from each sublibrary, each aliquot containing approximately 1000 beads, was added to a well of a 24-well polystyrene multi-well plate. From 1.5 to 2 ml Superblock (Pierce Chemical Company, Rockford, Ill.) reagent, 0.1% gelatin (fish skin gelatin, Sigma Chemical Company, St. Louis, Mo.), or 1% (w/v) bovine serum albumin (BSA, Sigma Chemical Company) in phosphate buffered saline (PBS), pH 7.4, was added to each well, and the plates were incubated for one to two hours at room temperature (RT), with periodic or continuous mixing by gentle rocking. The protein to be tested for binding was diluted in Superblock or 0.1% gelatin-PBS to give a final concentration of about 10.sup.-6 to 10.sup.-8 M. The diluted protein solution was incubated with the D-peptide-beads for one to two hours at RT. Following the incubation, the protein solution was removed and the beads washed three times with PBS. In the second wash, PBS was left on the beads for about 30 minutes to allow dissociation of weakly binding protein.

After washing with PBS, an agent for detecting bound protein was added. In some cases, the test protein was labeled with alkaline phosphatase (AP) and no secondary detection agent was required. In other cases, the test protein was labeled with biotin using the biotinylating reagent NHS-LC-biotin (Pierce Chemical Company) according to the supplier's instructions. Biotin-labeled protein was detected using AP conjugated to neutravidin (Pierce Chemical Company). Another means of detecting bound biotinylated proteins used AP-conjugated anti-biotin antibody reagent, which detects bound biotinylated protein on the beads. In other instances, the detection reagent was an AP-labeled antibody to the protein. The detection agents were generally incubated with the beads for 30 minutes, after which the beads were washed three times with a Tris-buffered saline solution (pH 7.5), with the second wash being left in contact with the beads for 30 minutes. One-step NBT/BCIP (nitro-blue tetrazolium chloride/5-bromo-4-chloro-3′-indolyphosphate p-toluidine salt) (Pierce Chemical Co.) was then added and the beads observed under a low power microscope until some of the beads had turned a dark purple to purple-black (dark purple-black) color. In the presence of AP enzyme, the phosphoryl group from BCIP is hydrolyzed and the BCI product reacts with NBT which then forms NBT-formazan. The NBT-formazan forms a purple-black precipitate on the beads to which the AP is attached. The beads were then washed with PBS twice, followed by a 1% acetic acid wash, and finally, water. The Fast Red TR/AS-MX substrate kit (Pierce Chemical Co.), which yields a bright red precipitate on beads positive for AP, was used in one experiment. The latter dye-precipitate can be removed by washing the beads with ethanol.

Dark purple-black beads, or bright red beads (when the Fast Red substrate was used), were removed using a small bore pipette and subjected to amino acid sequence analysis performed at the Core Laboratories of Louisiana State University Health Sciences Center. The sequences obtained were essentially unequivocal. Because the five sublibraries were kept separate, the first residue at the amino-terminus was known. For all D-peptides in the library, the sixth amino acid is G because G was coupled to the TentaGel beads. For purposes of reporting the D-peptide sequences, the sixth residue (G) is not reported.

Binding of Lectins PSA (Pisum sativum, Garden Pea Lectin) and ConA (Canavalia ensiformis, Jack Bean Lectin) to D-Peptide-Beads

The lectins as conjugated with AP (AP-PSA and AP-ConA) were purchased from EY Laboratories (San Mateo, Calif.). The lectins were incubated with the F and Y sublibraries by the procedure outlined above. The number of purple-black beads and the number of total beads were counted in each incubation well, and the percent positive beads was calculated. The approximate number of positive sequences was calculated based on 625 different D-peptide sequences each in the F- and Y-sublibraries (Table 1).

The relatively low percentage of positives obtained suggests that the binding between the D-peptides of the F and Y sublibraries and the lectins was selective. If proteins bound to the beads only due to the hydrophobicity of the D-peptide sequences, one would have expected to obtain a high percentage of positives. On the other hand, if the proteins had failed to bind to any of the D-peptide sequences, one might conclude that D-peptides do not fit into the lectin binding sites or to other surface areas of the lectin proteins. Instead, the results showed that the frequency of binding of the lectins was selective for a small percentage of the D-peptide sequences. Control experiments showed that the AP enzyme was not responsible for binding to D-peptide sequences.

The amount of protein binding to a bead was calculated to be about 5 pmoles protein/bead, based on the following assumptions: (I) AP-PSA and AP-ConA were added in a one ml volume to the beads and at a concentration of 0.1 μM; (2) the Kd for the D-peptide sequence and lectin complexes is assumed to be about 0.1 μM; (3) one-half of the total AP-ConA or AP-PSA protein is bound at equilibrium; and (4) an average of about 10 beads out of a 1000 are positive.

Cross-Reactivity of D-Peptide-Beads for AP-ConA and AP-PSA

To evaluate the ability of particular D-peptides to bind to both AP-ConA and AP-PSA, the F- and Y-sublibraries were incubated with either AP-ConA or AP-PSA. Positive beads were detected using the Fast Red TR/AS-MX substrate. The positive beads were removed, and the dye washed from the beads using ethanol. The original AP-ConA positive beads were then incubated with AP-PSA, and the original AP-PSA positive beads were incubated with AP-ConA. Positive beads were then detected using the NBT/BCIP dye reagent and the number of positive beads (purple-black color) was determined. Of 11 beads tested from the Y-sublibrary that were initially positive for AP-ConA binding, 3 (27%) were positive for AP-PSA binding. One of 9 (11%) Y-sublibrary initially positive for binding of AP-PSA was positive for AP-ConA binding. Of 26 beads from the F-sublibrary originally positive for AP-PSA, 8 (31%) were positive for binding of AP-ConA. Neither of the two beads from the F-sublibrary that were positive for AP-ConA binding bound to AP-PSA. Of the total beads tested (48), 12 (25%) were cross-reactive for both lectins. Thus, for lectins that share binding specificities for similar carbohydrate structures, certain D-peptide sequences may exhibit cross-reactive binding activities. ConA and PSA lectins have specificity for structures containing mannose in an α-anomeric glycosidic linkage at the non-reducing termini of oligosaccharides. It is therefore not surprising that certain of the D-peptides to which the lectins bind are the same, and that certain D-peptides may bind to more than one lectin.

Competitive Binding for Lectins Between D-Peptide Beads and the Carbohydrate Ligand

To test whether the lectins bind to D-peptide sequences through their carbohydrate binding sites, the D-peptide-beads of the F- and Y-sublibraries were incubated with AP-ConA in the presence and absence of 10 mM concentration of α-methyl-mannoside. The beads were then incubated with NBT/BCIP reagent. In the absence of a α-methyl-mannoside, 7.9% and 5.7% of D-peptides of the F- and Y-sublibraries, respectively, bound ConA. When incubated with the D-peptides in the presence of α-methyl-mannoside, ConA bound to 4.0% and 1.2% of the D-peptides in the F- and Y-sublibraries, respectively. The results suggest that approximately half of the positive D-peptide sequences in the F-sublibrary and a fifth of the positive D-peptides in the Y sublibrary D-peptide bind to the same binding site as that to which α-methyl-mannoside binds.

In an additional experiment, the F- and Y-sublibrary beads were first incubated with AP-ConA in the presence of α-methyl-mannoside yielding 2.1% of the Phe and 1.1% of the Tyr beads as positive. Those beads were removed from the incubation wells and the beads further incubated with AP-ConA without added α-methyl-mannoside. After the substrate NBT/BCIP was added, 3.6% of the Phe and 5.6% of the Tyr sublibraries turned dark purple-black again illustrating that a portion of the D-peptide sequences in each sublibrary were binding to the carbohydrate binding site of the ConA lectin.

Binding of Chicken Antibody and a Lectin to D-peptides

An affinity-purified chicken antibody developed against an antigen including an α-Gal epitope (Cook et al., J Biosci.& Bioeng., 91, 305-310, 2001) and a biotinylated lectin that binds to the same epitope, GS 1, B4 isoform (Murphy and Goldstein, J. Biol. Chem., 252, 4739-4742, 1977) were tested for binding to the A- and G-sublibraries. Binding of chicken antibody to beads was detected using an AP-labeled secondary antibody to chicken IgY. Binding of the lectin to beads was detected using AP-neutravidin. The chicken antibody and lectin were incubated with the beads at a concentration of 50 μg/ml, about 0.3 μM and 0.44 μM, respectively. The percentage of D-peptides binding to the antibody or lectin was determined as described herein.

These results (Table 2) show that an antibody to a carbohydrate epitope, as well as a lectin with a binding site to the same carbohydrate epitope, exhibit specificity in binding to the D-peptide sequences. Furthermore, the results show that a lectin with reactivity to a carbohydrate epitope different from that of ConA and PSA, exhibits binding to D-peptide sequences.

Binding Specificities of Two Additional Antibodies Reactive with Carbohydrate Epitopes to D-Peptide Sequences

A biotinylated mouse IgM monoclonal antibody to a Ley/H carbohydrate epitope (Holloran et al., J. Immunol., 164, 4868-4877, 2000) or affinity-purified human anti-αGal antibody, (Fryer et al., Xenotransplantation, 56:98-109, 1999) were incubated with D-peptides from the A-, G-, F-, Y-, and W-sublibraries. Binding to the D-peptides was detected using AP-labeled anti-mouse IgM reagent (Sigma) or AP-labeled anti-human Ig reagent (Sigma Chemical Co.). The percentage of D-peptides binding to the antibodies are shown in Table 3.

The results show that two additional anti-carbohydrate antibodies exhibit selective binding to the D-peptide beads. The mouse anti-Ley/H antibody was reactive with D-peptide sequences of the Y and W sublibraries. The antibody also bound D-peptides from the G sublibrary, but binding did not exceed background (i.e., AP-labeled anti-mouse IgM reagent bound to the same number of sequences in the presence and absence of anti-Ley/H antibody). The human anti-αGal antibody appeared to bind to D-peptide sequences of the A and G sublibraries; the AP-labeled anti-human Ig reagent only bound to one sequence of the W sublibrary. Thus, the D-peptide sequences appear to be specific for another form of anti-αGal antibody (human) compared to the chicken anti-αGal antibody in the previous example. It was not determined whether the human and chicken anti-αGal antibodies bound to the same D-peptide sequences on the TentaGel beads.

Preparation of Toxins

In the Examples that follow, several toxins were screened for the ability to bind to D-peptide sequences in the D-peptide bead library. The toxins include the neurotoxin component of the botulinum toxins, the cell binding B subunit of the cholera toxin, the protective antigen portion of the anthrax toxin, and the cell binding component of the ricin toxin. These toxins are particularly important because of their potential for use in biological warfare agents (J. Am. Med. Assoc., vol. 278, no. 5, Aug. 6, 1997).

The neurotoxin components of the A, B and E serotypes of the botulinum toxins, and the botulinum type B complex form (designated BoNT/A, BoNT/B, BoNT/E and BotBcomp, respectively) were purified by methods described by Tse et al. (Eur. J. Biochem, 122, 493-500, 1982) and Moberg and Sugiyama (Appl. Environ. Microbiol., 35, 878-880, 1987). The two forms of the ricin toxin (RCA60 and RCA120) were purchased from Sigma Chemical Co. (St. Louis, Mo.). The cholera toxin B subunit was purchased from List Biological Laboratories, Campbell, Calif. The protective antigen (PA) component of the anthrax toxin was kindly supplied by the United States Army Medical Research Institute of Infectious Diseases.

Frequency of Binding of BoNT/A and BoNT/B to D-Peptide Beads

Biotinylated BoNT/A and BoNT/B neurotoxins were incubated with the five sublibraries of D-peptide beads, and binding detected using the AP-neutravidin reagent, as described above. The frequencies of strong positive (purple-black) beads were determined.

The results (Table 4) indicate that the frequency of positives diminishes as the concentration of the toxin incubated with the beads is decreased below the sensitivity of detection. For example, binding of BoNT/A to D-peptides in the G sublibrary was detectable at BoNT/A concentrations of 667 nM and 67 nM, whereas binding of BoNT/A to D-peptides in the A and F sublibraries was detectable only at a BoNT/A concentration of 667 nM. The selectivity of the D-peptides for the toxins is suggested by the low frequencies of binding. The higher binding frequencies observed for the BoNT/B toxin may be due to differential biotinylation of BoNT/B and BoNT/A, to an effect on the BoNT/A activity due to biotinylation, or due to the greater activity of the particular purified preparations used in the screening assays.

Frequencies of Binding of Ricin Toxin (RCA60), Anthrax Protective Antigen (PA) and Cholera Toxin B Subunit (CT) to D-Peptide Beads

The RCA60 form of the ricin toxin, the PA protein and the B subunit of the cholera toxin were biotinylated, incubated with the D-peptide library beads, and binding detected using the AP-neutravidin reagent. Numbers of positive beads were counted and the frequencies calculated.

The results showed selective binding of the D-peptide sequences to each toxin component tested (Table 5).

Additional binding studies were performed with the BoNT/E toxin, with the RCA120 form of ricin, and with the botulinum B complex toxins (BotB complex).

Sequences of Positive Beads from the Binding Assays with the Various Proteins

Positive beads identified in binding assays were selected at random and the amino acid sequences determined for the individual beads. The sequences are shown in Table 6.

Of the total sequences obtained, 90% contain three or four aromatic D-amino acids. Of those sequences identified from the G and A sublibraries (i.e., D-peptides with G or A residues at the amino-terminus), 89% contained three or four aromatic D-amino acids. One sequence, GFYFF, was identified as binding to ConA, BoNT/B and RCA60. Another sequence, GYFFY, was identified as binding to BoNT/A and RCA60. A third sequence, AFYYF, was identified as binding to RCA60, BoNT/A and GS1-B4. In two instances, the same sequence was identified as binding to a particular protein: GFFYF for BoNT/A and WAFFF for RCA60.

Sequences of Positive Beads from Binding Studies Using TNFα and TGFβ1

TNFα and TGFβ1 obtained from commercial suppliers were incubated with the D-peptide library beads using the procedures described above and binding of the proteins detected using commercially available monoclonal and polyclonal antibody antibodies. Positive beads from the TNFα incubations with the F, Y and W sublibraries were removed and sequenced; positive beads from the incubation with TGFβ1 from the F sublibrary were removed and sequenced. The sequences are listed in Table 7.

The sequence YFAFF from the TNFα screen was found on four of the six Y sublibrary beads sequenced, and is the same sequence found as binding BoNT/A. Both beads sequenced from the F-sublibrary of the TNFα binding study had the identical sequence FFFAF. Two of the 27 total sequences (7%) contained two aromatic D-amino acids; six (22%) contained three aromatic D-amino acids; 17 (63%) contained four D-amino acids; and two (7%) contained five aromatic D-amino acids.

Microplate Assay to Determine Protein Binding to D-Peptide Sequences

Certain D-peptide sequences identified above as binding to proteins were synthesized with 3 or 4 D-lysine (K) residues at the carboxyl-terminus in order to increase solubility of the D-peptides in aqueous solutions. The D-lysine-containing D-peptides were covalently coupled to maleic anhydride-coated 96-well polystyrene plates (Pierce Chemical Co.) and the wells were backcoated. Coupling of the D-peptide occurs predominantly through the D-K amino groups and the majority of the D-peptides would then project from the walls of the plate into the solvent, mimicking the presentation of D-peptides on the TentaGel beads. Proteins were added to the D-peptide-coated wells at various concentrations and incubated for at least one hour to allow equilibrium of binding to occur. The wells were washed several times with PBS, and the relative amounts of protein bound were determined. Usually the proteins were biotinylated and the relative amounts of protein bound determined by adding AP-neutravidin and measuring bound AP by incubating with p-nitrophenyl-phosphate and measuring p-nitro-phenol calorimetrically. Maximum binding of proteins was established for the greater amounts of proteins added to the wells coated with particular D-peptides. Background binding for any protein was determined for wells not coated with D-peptide or wells lacking the protein incubation but with addition of the AP-neutravidin reagent. The dissociation equilibrium constant (Kd) may be estimated from the amount of protein added to the D-peptide coated wells that resulted in half maximal binding.

The D-peptides used to coat wells, the concentration of toxin at which saturation of binding was obtained, and the Kd estimates obtained for the proteins bound were as follows (Table 8).

The results indicate that the D-peptides have high binding affinities for the various toxins tested. It is of interest to note that the D-peptide sequence GFFFY, was identified as binding to the BoNT/B neurotoxin and the sequence in this binding assay bound both BoNT/B and BoNT/A neurotoxins as well as the BotB complex. The sequences GFGWY and GAFFW were also identified from D-peptide beads incubated with the BoNT/B neurotoxin and those D-peptides bound both the BoNT/A and BoNT/B neurotoxins. These results support that several of the D-peptide sequences will exhibit cross-reactivities to the structurally similar botulinum toxins, and that any one of such D-peptides, or mixture of D-peptides, may be useful for neutralizing the toxic effects of the several serotypes of the toxins and the Bot complex form of the toxin.

Test for Possible Toxicities of D-Peptides

Potential toxicities of D-peptides to be tested for the ability to neutralize toxins in animals was evaluated by injecting the D-peptides into mice intravenously (iv) or intraperitoneally (ip) and observing the animals over time for signs of toxicity (Table 9).

In experiments 1 and 2, the mice exhibited no apparent toxicity (e.g., lethargy or ruffled fur) over a five day time period of observation. In experiments 3 and 4, the mice appeared to exhibit lethargy for the first 1 to 2 hours following administration of the D-peptides, and then exhibited no apparent signs of toxicity and appeared normal for the remainder of the three-day observation period. In experiment 5, the mice initially exhibited lethargy, ruffled fur and isolationism, then appeared normal on the following day of observation.

Prolongation of Survival of Mice Injected with Botulinum Toxin Plus D-Peptides

Experiment 1 (Table 10). Two groups of five mice each were injected ip with 500×LD×.50 of BoNT/A neurotoxin alone or the same amount of neurotoxin plus a D-peptide mixture. The D-peptide mixture contained GYFFFKKK (263 μg), GFFYFKKK (500 μg), and GYFYFKKK (220 μg). Times to death for animals in each group were noted.

The animals alive at >300 minutes were dead the following morning.

The mean survival times for the five animals given BoNT/A only was 194.+-.29 (SEM) minutes (using 300 minutes for the one animal that survived for the initial five hours observation time). The mean survival time for the animals given BoNT/A plus the D-peptides was 269.+-.20 minutes (using 300 minutes for the two mice that survived for the initial five hours of observation time). The p value for the differences in survival times between the two groups by the Students t test was 0.14; the p value using the chi square test was 0.11.

The mean survival times of mice given a large dose of BoNT/A (equivalent to 500× the LD×50 the toxin) and treated with D-peptides was increased by at least 35%, relative to untreated mice given the same dose of BoNT/A.

Experiment 2 (Table 11). Experiment 1 was repeated using the same amounts of BoNT/A neurotoxin (mice injected with 500×LD₅₀ of the toxin). The D-peptide mixture comprised GYFFFKKK (310 μg), GFFYFKKK (382 μg), and GYFYFKKK (310 μg) with the neurotoxin. Times to death for animals in each group were noted.

Of the two mice that survived greater than the 330 minutes of initial observation time, one was dead the next morning and the other mouse survived.

The mean survival times of the animals given BoNT/A only was 135.+-.8.5 (SEM) minutes. The mean survival times of the mice given BoNT/A plus D-peptides was 278.+-.29 minutes (using 330 minutes as the survival times of the two mice that survived the initial 5.5 hour observation time). The p value for the difference in survival times was 0.01 using the Students t test and 0.009 using the chi square test.

The mean survival times of the group treated with BoNT/A and D-peptides was double that of the group treated with BoNT/A alone, and the differences were statistically significant.

Method for Reducing Toxicity of a Toxin

A method for reducing the toxicity of a toxin in a mammal exposed to the toxin includes delivering to the mammal a D-peptide(s) that binds to the toxin in an amount effective to reduce toxicity, wherein the D-peptide(s) comprises from three to seven D-amino acid residues, wherein at least three of the D-amino residues are independently selected from the group consisting of D-phenylalanine, D-tryptophan, and D-tyrosine.

The toxin is from the group including botulinum toxins, ricin toxins, cholera toxins, and anthrax toxins or toxin subcomponents.

The toxin may be BoNT/A and the D-peptide includes a pentapeptide core sequence from the group Xaa₁YFFF, Xaa₁FFYF, Xaa₁YFFY, Xaa₁YFYF, Xaa₁FFFF, Xaa₁FYYF, Xaa₁FFYF, FXaa₁FFF, YFXaa₁FF, wherein Xaa₁ is an amino acid of the D- or L-configuration from the group D, E, K, R, H, N, Q, C, S, T, G, A, V, L, I, M, and P.

The toxin may be BoNT/A and the D-peptide includes pentapeptide core sequence from the group GYFFF, GFFYF, GYFFY, GYFYF, AFFFF, AFYYF, AFFYF, FAFFF, and YFAFF.

The toxin may be BoNT/B and the D-peptide includes a pentapeptide core sequence from the group Xaa₁FWXaa₂Y, Xaa₁FXaa₂WY, Xaa₁Xaa₂FFW, Xaa₁FFFY, Xaa₁FYFF, Xaa₁FYFF, Xaa₁FFFY, FFFFXaa₁, YXaa₁YFF, and YXaa₁FFY, wherein Xaa₁ and Xaa₂ are amino acids of the D- or L-configuration from the group D, E, K, R, H, N, Q, C, S, T, G, A, V, L, I, M.

The D-peptide includes a pentapeptide core sequence from the group GFWGY, GFGWY, GAFFW, GFFFY, GFYFF, AFYFF, AFFFY, FFFFG, YAYFF, and YAFFY.

The toxin may be BoNT/E and the D-peptide includes a pentapeptide core sequence from the group Xaa₁FFXaa₂Xaa₃ and Xaa₁WYFF, wherein Xaa₁, Xaa₂, and Xaa₃ are amino acids of the D- or L-configuration independently selected from the group consisting of D, E, K, R, H, N, Q, S, T, G, A, V, L, I, M, and P. The D-peptide includes a pentapeptide core sequence from the group GFFGA and GWYFF.

The toxin may be BotB complex and the D-peptide includes a pentapeptide core sequence from the group Xaa₁FXaa₂FF, Xaa₁YXaa₂FF, Xaa₁FFYXaa₂, Xaa₁FFXaa₂F, Xaa₁Xaa₂Xaa₃-YY, and Xaa₁Xaa₂Xaa₃FF wherein Xaa₁, Xaa₂, and Xaa₃ are amino acids of the D- or L-configuration independently selected from the group D, E, K, R, H, N, Q, C, S, T, G, A, V, L, I, M, and P.

The D-peptide includes a pentapeptide core sequence from the group GFGFF, GYGFF, GFFYG, GFFGF, AAGYY, and AAAFF.

The toxin may be RCA60 and the D-peptide includes comprises a pentapeptide core sequence from the group of Xaa₁FYWF, Xaa₁Xaa₂FYY, Xaa₁YYFY, Xaa₁FYFF, Xaa₁YFFY, Xaa₁FYXaa₂Y, Xaa₁FYYF and WXaa₁FFF, wherein Xaa₁ and Xaa₂ are amino acids of the D- or L-configuration independently selected from the group D, E, K, R, H, N, Q, C, S, T, G, A, V, L, I, M, and P.

The D-peptide includes a pentapeptide core sequence from the group GFYWF, GGFYY, GYYFY, GYFFY, GYFFY, AFYAY, AFYYF and WAFFF.

The toxin may be RCA120 and the D-peptide includes a pentapeptide core sequence from the group Xaa₁FFEXaa₂ and Xaa₁YYYY, wherein Xaa₁ and Xaa₂ are amino acids of the D- or L-configuration independently selected from the group D, E, K, R, H, N, Q, S, T, G, A, V, L, I, M, and P.

The D-peptide includes a pentapeptide core sequence selected from the group GFFFA and AYYYY.

The toxin may be cholera toxin and the D-peptide includes a pentapeptide core sequence from the group FXaa₁WFF and WXaa₁FWXaa₂, wherein Xaa₁ and Xaa₂ are amino acids of the D- or L-configuration independently selected from the group D, E, K, R, H, N, Q, C, S, T, G, A, V, L, I, M and P.

The D-peptide includes a pentapeptide core sequence selected from the group FAWFF and WAFWA.

The toxin anthrax protective antigen and the D-peptide includes a pentapeptide core sequence from the group YGYYA and WFXaa₁FXaa₂ wherein Xaa₁ and Xaa₂ are amino acids of the D- or L-configuration independently selected from the group D, E, K, R, H, N, Q, C, S, T, G, A, V, L, I, M and P.

The D-peptide includes a pentapeptide core sequence from the group YGYYA and WFAFG.

A D-peptide comprising a pentapeptide sequence selected from the group consisting of Xaa₁YYFF, Xaa₁FYFF, Xaa₁YFFF, Xaa₁FFYF, Xaa₁YFFY, Xaa₁YFYF, Xaa₁FFFF, Xaa₁FYYF, FXaa₁FFF, YFXaa₁FF, Xaa₁FWXaa₂Y, Xaa₁FXaa₂WY, Xaa₁Xaa₂FFW, Xaa₁FFFY, FFFFXaa₁, YXaa₁YFF, YXaa₁FFY, Xaa₁FFXaa₂Xaa₃, Xaa₁WYFF, Xaa₁FXaa₂FF, Xaa₁YXaa₂FF, Xaa₁FFYXaa₂, Xaa₁FFXaa₂F, Xaa₁Xaa₂Xaa₃YY, Xaa₁Xaa₂Xaa₃FF, Xaa₁FYWF, Xaa₁Xaa₂FYY, Xaa₁YYFY, Xaa₁FYXaa₂Y, WXaa₁FFF, Xaa₁FFFXaa₂, Xaa₁YYYY, FXaa₁WFF, WXaa₁FWXaa₂, WFXaa₁FXaa₂, FWXaa₁FF, FXaa₁FFY, Xaa₁Xaa₂WXaa₃Y, FFWXaa₁Y, FXaa₁Wxaa₂Xaa.sub-0.3, YYXaa₁YY, FFFXaa₁F, YFYFXaa₁, YWXaa₁FF, WXaa₁Yxaa₂F, WXaa₁YFXaa₂, WXaa₁FFXaa₂, FFFXaa₁W, FWFXaa₁Xaa₂, FYXaa₁YF, FWXaa₁Xaa₂Xaa₃, FXaa₁YYW, FXaa₁YYXaa₂, FWXaa₁WY, FFWYW, FXaa₁Xaa₂FXaa₃, FYWXaa₁Y, FYWXaa₁W, FXaa₁YFXaa₂, FWWYF, FYYYXaa₁ and FFXaa₁WW wherein Xaa₁, Xaa₂, and Xaa₃ are amino acids of the D- or L-configuration independently selected from the group consisting of D, E, K, R, H, N, Q, C, S, T, G, A, V, L, I, M, and P.

The D-peptide wherein the core pentapeptide is selected from the group consisting of GYYFF, GFYFF, GYFFF, GFFYF, GYFFY, GYFYF, AFFFF, AFYYF, AFFYF, FAFFF, YFAFF, GFWGY, GFGWY, GAFFW, GFFFY, AFYFF, AFFFY, FFFFG, YAYFF, YAFFY, GFFGA, GWYFF, GFGFF, GYGFF, GFFYG, GFFGF, AAGYY, AAAFF, GFYWF, GGFYY, GYYFY, AFYAY, WAFFF, GFFFA, AYYYY, FAWFF, WAFWA, YGYYA, WFAFA, AFFFA, FWAFF, FAFFY, GAWAY, FFWGY, FAWGA, YYAYY, FFFAF, YFYFA, YWAFF, FFFGW, FWFGA, FYGYF, FWAAA, FAYYW, FGYYG, FWAWY, FFWYW, FAAFG, FYWAY, FYWGW, FAYFG, FYYYA, FWGFF, and FFAWW.

### Materials and Methods

The calculations of the percents of peptides in the combinatorial libraries containing 3 to 7 amino acid residues, the tri- to hepta-peptide combinatorial libraries, are obtained using simple logic and math. In order to do the calculations, one first determines the number of “permutations” in peptides that contain 3 or more aromatic residues (D-phenyalanine, D-tyrosine and D-tryptophan). For example, using the pentapeptide combinatorial library, one permutation is 3 aromatic residues in one of the peptide sequences at positions 1, 2, 3 of the 5 positions of the peptide. Another permutation would be 1, 2, 4. The rest of the permutations for 3 aromatic residues are: 1, 2, 5; 1, 3, 4; 1, 3, 5; 1, 4, 5; 2, 3, 4; 2, 3, 5; 2, 4, 5; and 3, 4, 5. So there are a total of 10 permutations for 3 aromatic residues in the pentapeptide library constructed as described herein.

For peptides in the pentapeptide combinatorial library having 4 aromatic residues, the permutations are: 1, 2, 3, 4; 1, 2, 3, 5; 1, 2, 4, 5; 1, 3, 4, 5; and 2, 3, 4, 5. So there are 5 permutations for 4 aromatic residues in the pentapeptide library as constructed.

For peptides in the pentapeptide library containing 5 aromatic residues, there is one permutation: 1, 2, 3, 4, 5. Thus all peptides in the pentapeptide library containing 5 aromatic residues have D-phenylalanine, D-tyrosine or D-tryptophan at each of the 5 positions in the sequences.

For each position that has an aromatic residue, that residue can be one of the 3 aromatics specified. So, for the permutation of positions 1, 2, 3, each containing one of the 3 aromatics, and in the pentapeptide library, there are 3 to the 3^{rd} power number=27 different peptide sequences. The other two positions, positions 4 and 5, have either D-alanine or glycine. Thus the number of pentapeptides having 3 aromatics at positions 1, 2, 3, can also have an additional 2 to the 2^{nd} power=4 different possible sequences for those pentapeptides containing aromatics at positions 1, 2, 3 and either D-alanine or glycine at positions 4 and 5. So there are 27×4=108 different peptide sequences that contain 3 aromatic residues at positions 1, 2, 3. Because there are 10 permutations for positions of any 3 of the aromatics in the 5 positions of the pentapeptides in the combinatorial library, there are a total of 10×108=1080 peptides in the pentapeptide library that contain 3 aromatic residues.

For the pentapeptides containing 4 aromatic residues, there will be 3 to the 4^{th} power (i.e., 4 positions)=81×2 (D-alanine or glycine at the other position) for each permutation. Five permutations×162=810 sequences in the pentapeptide combinatorial library will have 4 aromatic residues.

For the pentapeptides that contain 5 aromatic residues, i.e., an aromatic residue at each of the 5 positions, there are 3 to the 5^{th} power=243 different sequences in the pentapeptide library.

So the total number of peptides containing 3 or more aromatic residues in the pentapeptide combinatorial library is 1080 plus 810 plus 243=2,133; 2,133 divided by 3,125 (the total number of peptides in the pentapeptide library)=0.68256=68.256%.

The hexapeptide combinatorial library contains 20 permutations of sequences with 3 aromatic residues, 15 permutations with 4 aromatic residues, 6 permutations with 5 aromatic residues, and 1 permutation with 6 aromatic residues; the remaining positions occupied by either D-alanine or glycine. So the total number of peptides in the hexapeptide library is 3 to the 3^{rd} power×2 to the 3^{rd} power×20 permutations, plus 3 to the 4^{th} power×2 to the 2^{nd} power×15 permutations, plus 3 to the 5^{th} power×2×6 permutations, plus 3 to the 6^{th} power×1 permutation which totals to 12,825 different hexapeptide sequences containing 3 or more aromatic D-amino acid residues. The total number of sequences in the hexapeptide combinatorial library is 15,625 (5 to the 6^{th} power); divided into 12,825=0.8208 or 82.08% of the sequences contain 3 or more aromatic D-amino acid residues.

For the heptapeptide combinatorial library, there are 35 permutations of sequences with 3 aromatic D-amino acid residues, 30 permutations of sequences with 4 aromatic residues, 20 permutations with 5 aromatic residues, 7 permutations with 6 aromatic residues, and 1 permutation with 7 aromatic residues. Doing the math yields 66,393 different sequences with 3 or more aromatic D-amino acid residues in the heptapeptide combinatorial library. This latter number divided by the total number of different sequences is 0.8498 so 84.98% of all sequences contain 3 or more aromatic D-amino acid residues.

For the tetrapeptide combinatorial library, the 3 aromatic amino acids plus glycine are used to make the library so the total number of sequences in the library=256. The number of sequences containing 3 or 4 aromatic residues is calculated from the fact that there are 4 permutations for 3 aromatics in different peptide sequences and 1 permutation for 4 aromatic residues=189 sequences containing 3 or 4 aromatic residues. 189 divided by 256=0.738 or 73.8% of all tetrapeptide sequences contain 3 or 4 aromatic residues.

For the tripeptide combinatorial library, there are 3 positions and each has one of the 3 aromatic D-amino acid residues so all 27 different sequences contain 3 aromatic residues.

TABLE 1
Binding of AP-ConA and AP-PSA Lectins
to F and Y Sublibrary D-peptide Beads

| | | | Number of positive beads/ |
|---|---|---|---|
| | | Percent positive | 625 possible sequences |
| AP-ConA | F Sublibrary | 2.1 | 13 |
| | Y Sublibrary | 1.3 | 8 |
| AP-PSA | F Sublibrary | 2.6 | 16 |
| | Y Sublibrary | 1.6 | 10 |

TABLE 2
Frequencies of Binding of Chicken Anti-αGal Antibody
and the Lectin GS1-B4 to the G- or
A-Sublibraries of D-peptide Beads

| | | Number of positive beads |
|---|---|---|
| | Percent positive | 625 possible sequences |
| Chicken anti-αGal | | |
| A sublibrary | 0.07 | 1 |
| G sublibrary | 0.6 | 4 |
| GS1-B4 lectin | | |
| A sublibrary | 2.9 | 18 |
| G sublibrary | 3.8 | 24 |

TABLE 3
Frequencies of Binding of Two Anti-carbohydrate Antibodies
to the D-peptide Beads

| | | Percent | Number of positive beads/ |
|---|---|---|---|
| | Sublibrary | positive | 625 possible sequences |
| Mouse anti-Ley/H | A | 0 | 0 |
| | G | 0.4 | 3 |
| | F | 0 | 0 |
| | Y | 0.5 | 3 |
| | W | 0.5 | 3 |
| AP-labeled anti-mouse | A | 0 | 0 |
| IgM reagent | G | 0.5 | 3 |
| | F | 0 | 0 |
| | Y | 0 | 0 |
| | W | 0 | 0 |
| Human anti-αGal | A | 0.1 | 1 |
| | G | 0.2 | 1 |
| | F | 0 | 0 |
| | Y | 0 | 0 |
| | W | 0 | 0 |
| AP-labeled anti-human | A | 0 | 0 |
| IgG reagent | G | 0 | 0 |
| | F | 0 | 0 |
| | Y | 0 | 0 |
| | W | 0.1 | 1 |

TABLE 4
Frequencies of Binding of BoNT/A and BoNT/B Neurotoxins
to the D-peptide Beads

| | | | | Number |
|---|---|---|---|---|
| | | Conc^{#}, | Percent | of positive beads/625 |
| | Sublibrary | (ug/ml, nM) | positive | possible sequences |
| BoNT/A | G | 100, 667 | 0.2 | 1 |
| | G | 10, 67* | 0.3 | 2 |
| | G | 10, 67* | 0 | |
| | G | 10, 67* | 0.2 | 1 |
| | G | 1, 6.7 | 0 | |
| | A | 100, 667 | 0.2 | 1 |
| | A | 10, 67* | 0 | |
| | A | 10, 67* | 0 | |
| | A | 10, 67* | 0 | |
| | A | 1, 6.7 | 0 | |
| | F⁺ | 100, 667 | 0.1 | 1 |
| | Y⁺ | 100, 667 | 0 | |
| | W⁺ | 100, 667 | 0 | |
| BoNT/B | G | 10, 67 | 2.3 | 14 |
| | G | 5, 33 | 0.5 | 3 |
| | G | 1, 6.7 | 1.0 | 6 |
| | G | 0.1, 0.67 | 0 | |
| | A | 10, 67 | 1.6 | 10 |
| | A | 5, 33 | 0 | |
| | A | 1, 6.7 | 0.1 | 1 |
| | A | 0.1, 0.67 | 0 | |
| | F | 10, 67 | 0.7 | 4 |
| | F | 5, 33 | 0 | |
| | F | 1, 6.7 | 0.1 | 1 |
| | F | 0.1, 0.67 | 0 | |
| | Y | 10, 67 | 0.6 | 4 |
| | Y | 5, 33 | 0 | |
| | Y | 1, 6.7 | 0.3 | 2 |
| | Y | 0.1, 0.67 | 0 | |
| | W | 10, 67 | 1.0 | 6 |
| | W | 5, 33 | 0 | |
| | W | 1, 6.7 | 0 | |

^{#}Concentration of toxin in the incubation with the D-peptide beads.
*Assay for binding to the beads was repeated 3 times at the 10 ug/ml concentration.
⁺There was no binding of the BoNT/A toxin to the D-peptide beads at the 10 and 1 ug/ml concentrations.

TABLE 5
Frequencies of Ricin (RCA60), Protective Antigen (PA)
and Cholera Toxin (CT) Binding to D-peptide Beads

| | | | | Number |
|---|---|---|---|---|
| | | Conc^{#}, | Percent | of positive beads/625 |
| | Sublibrary | (ug/ml, nM) | positive | possible sequences |
| RCA60 | G | 5, 83 | 0.5 | 3 |
| | A | ″ | 0.8 | 5 |
| | F | ″ | 2.8 | 18 |
| | Y | ″ | 1.4 | 9 |
| | W | ″ | 2.3 | 14 |
| PA | G | 23, 40 | 0.5 | 3 |
| | A | ″ | 0.4 | 3 |
| | F | ″ | 0.4 | 3 |
| | Y | ″ | 0.2 | 1 |
| | W | ″ | 0.2 | 1 |
| CT | G | | 0.3 | 2 |
| | A | | 0 | |
| | F | | 0.2 | 1 |
| | Y | | 0 | |
| | W | | 0.4 | 3 |

^{#}Concentration of the protein in the incubation with the D-peptides.

TABLE 6
Sequences of D-peptides binding
to tested lectins or toxins

| Lectin or Toxin | Sequences |
|---|---|
| ConA | GYYFF; GEYFF |
| BoNT/A | GYFFF; GFFYF; GEFYF; GYFFY; GYFYF |
| | AFFFF; AFYYF; AFFYF |
| | FAFFF |
| | YFAFF |
| BoNT/B | GFWGY; GEG WY; GAFFW; GFFFY; GFYFF |
| | AFYFF; AFFFY |
| | FFFFG |
| | YAYFF; YAFFY |
| BoNT/E | GFFGA; GWYFF |
| BotB complex | GFGFF; GYGFF; GFFYG; GFFGF |
| | AAGYY; AAAFF |
| RCA60 | GFYWF; GGFYY; GYYFY; GFYFF; GYFFY |
| | AFYAY; AFYYF |
| | WAFFF; WAFFF |
| RCA120 | GFFFA |
| | AYYYY |
| Cholera | FAWFF |
| Toxin | WAFWA |
| Protective | YGYYA |
| Antigen | WFAFG |
| (anthrax toxin) | |
| GS1-B4 lectin | AFYYF; AFFFA |
| | FWAFF; FAFFY |
| Human anti-αGal | GAWAY; FFWGY; FAWGA |
| Antibody | |
| Anti-Ley/H | YYAYY |
| antibody | |

TABLE 7
Sequences of D-peptides
Binding TNFα or TGFβ1

| | |
|---|---|
| TNFα: | FFFAF; FFFAF |
| | YFAFF; YFAFF; YFAFF; YFAFF; YFYFA; YWAFF |
| | WGYAF; WGYFA; WAFFA |
| TGFβ1 | FFFGW; FWFGA; FYGYF; FWAAA: FAYYW; FGYYG; |
| | FWAWY; FFWYW; FAAFG; FYWAY; FYWGW; FAYFG; |
| | FYYYA; FWGFF; FFAWW |

TABLE 8
Determination of Dissociation Constants
for D-peptides Binding to Various Toxins

| | | Saturation | Estimated |
|---|---|---|---|
| D-peptide Sequences* | | Concentration, | Kd, |
| used for Coating of Wells | Toxin Bound | nM | nM |
| GFYFF, AFYAY or | RCA60 | 100 | 20-25 |
| GFFFY | BoNT/A or | 3-4 | 1-2 |
| GFGWY or GAFFW | BoNT/A or | 3-4 | 0.5-1 |
| GFFFY | BotB complex | 2.3 | 0.022 |

*The D-peptides used for coating the wells of the microplates each had three D-K residues added to the carboxyl-terminus for solubility and coating purposes.

TABLE 9
D-peptides Used in Toxicity Studies

| | | | | | Concen- |
|---|---|---|---|---|---|
| Experi- | Number | Route of | | Amounts, | trations*, |
| ment | Of mice | injection | D-peptide(s)^{#} | ug | mM |
| 1 | 5 | iv | GFWGY | 50 | 0.025 |
| 2 | 3 | ip | GFWGY | 250 | 0.125 |
| 3 | 2 | ip | GFYFF, | 640 | 0.30 |
| | | | AFYAY, | | |
| | | | WAFFF | | |
| 4 | 2 | ip | GFFYF, | 430 | 0.21 |
| | | | GYFFY | | |
| 5 | 3 | ip | GYFFF, GFFYF, | 973 | 0.46 |

^{#}The D-peptides used each had three D-lysine residues added to the carboxyl-terminus.
*The concentrations were calculated assuming a 2-ml blood volume for the mice.

TABLE 10
Survival Times of Mice Injected with Toxin in the Presence or Absence of
D-peptides

Times to death in minutes

| | | BoNT/A plus |
|---|---|---|
| Animal number | BoNT/A group | D-peptides group |
| 1 | 140 | 193 |
| 2 | 142 | 260 |
| 3 | 191 | 290 |
| 4 | 198 | >300 |
| 5 | >300 | >300 |

The animals alive at >300 minutes were dead the following morning.

TABLE 11
Survival Times of Mice Injected with Toxin in the Presence or Absence of
D-peptides

Times to death in minutes

| | | BoNT/A plus |
|---|---|---|
| Animal number | BoNT/A group | D-peptides group |
| 1 | 117 | 193 |
| 2 | 121 | 231 |
| 3 | 137 | 309 |
| 4 | 138 | >330 |
| 5 | 165 | >330 |

I claim:

1. Combinatorial libraries comprising a plurality of D-peptides, wherein each D-peptide comprises from three to seven D-amino acid residues, wherein at least 68% of the D-peptide sequences comprise at least three amino acid residues independently selected from the group consisting of D-tryptophan, D-tyrosine, and D-phenylalanine.

2. The combinatorial library of claim 1, wherein each D-peptide is attached to a solid support.

3. The combinatorial library of claim 2, wherein the solid support is attached to a bead.

4. The combinatorial library of claim 1, wherein each peptide is attached to a microtiter plate.

5. A method for reducing the toxicity of a toxin in a mammal exposed to the toxin comprising delivering to the mammal a D-peptide(s) that binds to the toxin in an amount effective to reduce toxicity, wherein the D-peptide(s) comprises from three to seven D-amino acid residues, wherein at least three of the D-amino residues are independently selected from the group consisting of D-phenylalanine, D-tryptophan, and D-tyrosine.

6. A method of reducing binding of TNFα to a TNFα receptor comprising delivering to the mammal a D-peptide comprising a pentapeptide core selected from the group consisting of FFFXaa₁F, YFXaa₁FF, YFYFXaa₁, YWXaa₁FF, WXaa₁YXaa₂F, WXaa₁YFXaa₂ and WXaa₁FFXaa₂ wherein Xaa₁ and Xaa₂ are amino acids of the D- or L-configuration independently selected from the group consisting of D, E, K, R, H, N, Q, C, S, T, G, A, V, L, I, M and P.

7. The method of claim 6, wherein the D-peptide comprises a pentapeptide core sequence selected from the group consisting of FFFAF, YFAFF, YFYFA, YWAFF, WGYAF, WGYFA and WAFFA.

8. A method of reducing the binding of TGFβ1 to a TNFβ1 receptor comprising delivering to the mammal a D-peptide comprising a pentapeptide core selected from the group consisting of FFFXaa₁W, FWFXaa₁Xaa₂, FYXaa₁YF, FWXaa₁Xaa₂Xaa₃, FXaa₁YYW, FXaa₁YYXaa₂, FWXaa₁WY, FFWYW, FXaa₁Xaa₂FXaa₃, FYWXaa₁Y, FYWXaa₁W, FXaa₁YFXaa₂, FYYYXaa₁, FWXaa₁FF and FFXaa₁WW wherein Xaa₁, Xaa₂ and Xaa₃ are amino acids of the D- or L-configuration independently selected from the group consisting of D, E, K, R, H, N, Q, C, S, T, G, A, V, L, I, M and P.

9. The method of claim 8, wherein the D-peptide comprises pentapeptide core sequence selected from the group consisting of FFFGW, FWFGA, FYGYF, FWAAA, FAYYW, FGYYG, FWAWY, FFWYW, FAAFG, FYWAY, FYWGW, FAYFG, FYYYA, FWGFF and FFAWW.

10. A method of reducing inhibiting anti-Ley/H antibody binding to an Ley/H epitope comprising delivering to the mammal a D-peptide comprising a pentapeptide core selected from the group consisting of YYXaa₁YY wherein Xaa₁ is independently selected from a group consisting of D, E, K, R, H, N, Q, C, S, T, G, A, V, L, I, M and P, the latter amino acids being of D- or L-configuration.

11. The method of claim 10, wherein the D-peptide comprises a pentapeptide core sequence selected from the group consisting of YYAYY.

12. A method of reducing binding of an anti-αGal antibody to an αGal epitope comprising delivering to the mammal a D-peptide comprising a pentapeptide core selected from the group consisting of Xaa₁Xaa₂WXaa₃Y, FFWXaa₁Y and FXaa₁WXaa₂Xaa.₃ wherein Xaa₁, Xaa₂ and Xaa₃ are amino acids of the D- or L-configuration independently selected from the group consisting of D, E, K, R, H, N, Q, C, S, T, G, A, V, L, I, M and P.

13. The method of claim 12, wherein the D-peptide comprises a pentapeptide core sequence selected from the group GAWAY, FFWGY and FAWGA.

14. A D-peptide comprising a sequence of from three to seven D-amino acids, wherein at least three of the amino acid residues are independently selected from the group consisting of D-tryptophan, D-tyrosine, and D-phenylalanine.

15. A method for identifying D-peptides having the ability to bind to a pre-selected protein comprising contacting the protein with combinatorial libraries of D-peptides according to claim 1, detecting binding of the protein to one or more D-peptides in the combinatorial libraries, and identifying the D-peptides.

16. A method for making D-peptides that bind to pre-selected proteins, comprising contacting the library of claim 1 with the individual proteins, detecting binding of the proteins to one or more D-peptides, identifying the D-peptides, and synthesizing the D-peptides.
